(19) United States
(12) Patent Application Publication
(10) Pub. No.: US 2015/0308789 A1
(43) Pub. Date: Oct. 29, 2015

(54) DETECTION SYSTEM OF A METHOD OF PERFORMING A SHOOTING EXERCISE

(71) Applicant: FABBRUCA D'ARMI PIETRO BERETTA S.p.A., Gardone Val Trompia, BRESCIA (IT)

(72) Inventor: Eugenio RIGHI, Gardone Val Trompia, Brescia (IT)

(21) Appl. No.: 14/651,570

(22) PCT Filed: Oct. 24, 2013
(86) PCT No.: PCT/IB2013/059599

§ 371 Date: Jun. 11, 2015

(30) Foreign Application Priority Data
Dec. 14, 2012 (IT) BS2012A000179

(51) Int. Cl. F41G 3/26; A61B 5/00; F41A 33/00

(52) U.S. Cl. CPC F41G 3/26; F41A 33/00; A61B 5/00

(57) ABSTRACT

A detection system (1) of a method of performing a shooting exercise with a weapon (10) includes a weapon and at least one out of the weapon detector (100) and an item of clothing (30) having a physical state detector and/or motor detector (300). The system (1) includes a collection and storage unit (50) suitable for receiving and storing the information detected by the weapon detector (100) and/or by the physical state and/or motor detector (300).

The present invention relates to a detection system of a method of performing a shooting exercise with a weapon.

Specifically, in fact, said system is suitable for generating, receiving and storing possible information and data relative to the method of performing a shooting exercise with a weapon.

Said weapon is in general any weapon suitable for performing a shooting exercise at a fixed or moving target; preferably said weapons are firearms or cold weapons; preferably said weapon is a rifle, a pistol or even a bow or crossbow.

In a preferred embodiment, the targets may be fixed, at variable distances or moving, such as a clay pigeon, depending on the type of exercise and practice the user wishes to perform.

In the prior art, in order to practice shooting exercises, the user needing or wishing for feedback on the method of performing the exercise, was forced to train in the presence of a trainer who by observing him could indicate any issues related to his posture and/or to his movements, for instance whether sufficiently smooth or not.

Up to today therefore the user could never train alone, if he want feedback relative to performance of the shooting exercise which is not, clearly, the result of said exercise.

The purpose of the present invention is therefore to overcome said problem typical of the prior art.

Specifically, the detection system which the present invention relates to, sets out to provide a system suitable to permit the user to train independently in such a way as to have feedback on the method of performing a shooting exercise.

Such purpose is achieved by a system made according to claim 1.

Further characteristics and advantages of the present invention are protected by means of the dependent claims.

The characteristics and advantages of the system according to the present invention will, in addition, be evident from the description given below, by way of a non-limiting example, according to the appended drawings, wherein:

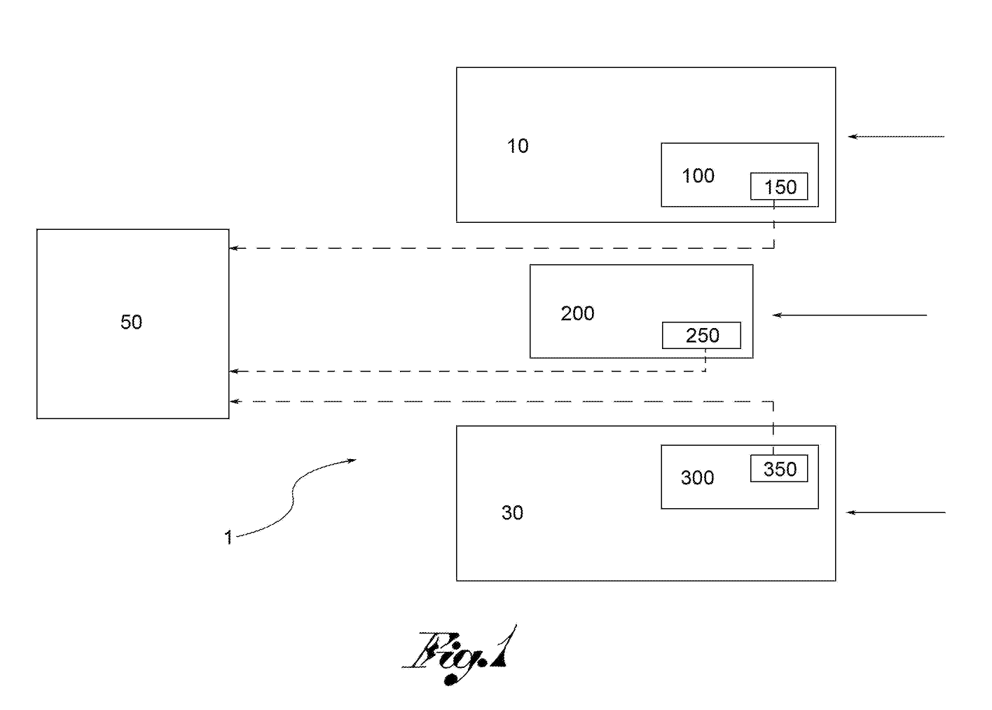

FIGS. 1a, 1b and 1c are three schematic diagrams of three logical diagrams showing some embodiments of the detection system of a method of performing a shooting exercise of the present invention.

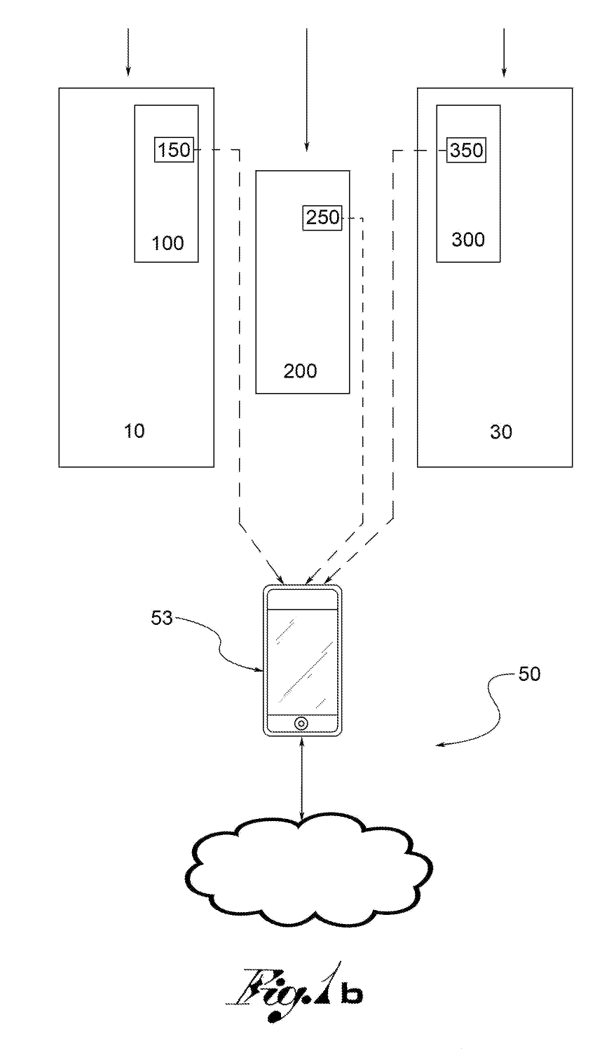

FIG. 2 shows a schematic diagram of a weapon comprising a series of weapon detection means comprised in the system which the present invention relates to;

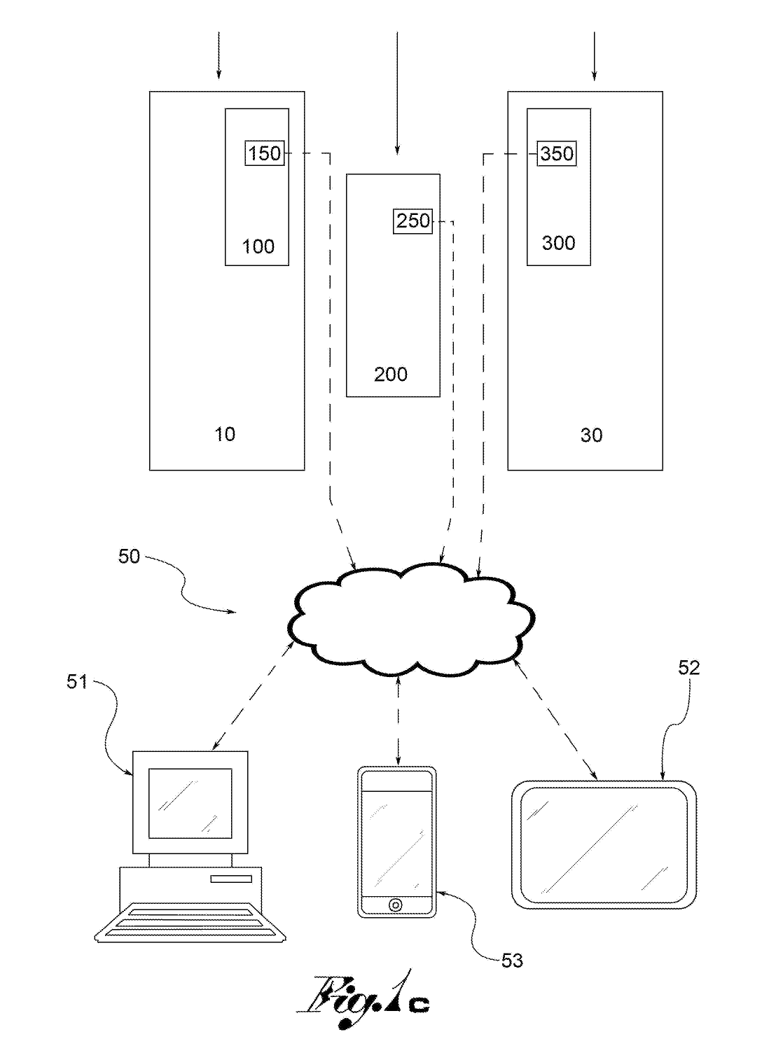

FIG. 3 shows a schematic diagram of an item of clothing, specifically, a T-shirt, comprising a series of physical state and/or motor detection means, comprised in a preferred embodiment of the system, which the present invention relates to;

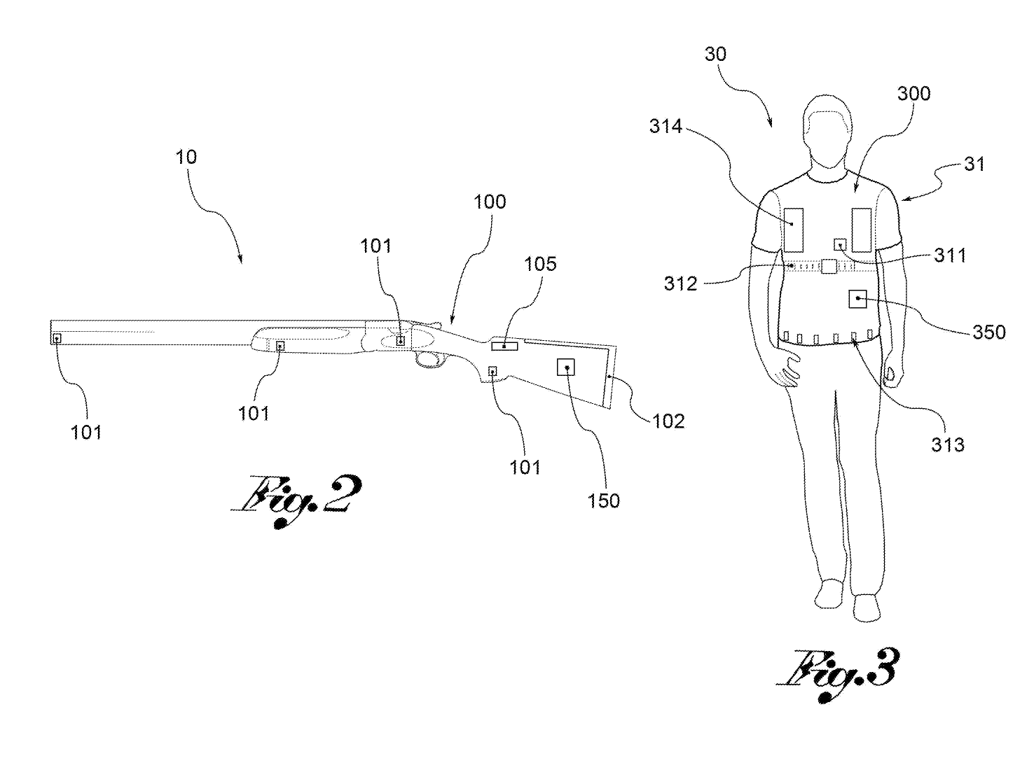

FIG. 4 shows a schematic diagram of an item of clothing, a coat, specifically a waistcoat, comprising a series of physical state and/or motor detection means, comprised in a preferred embodiment of the system which the present invention relates to;

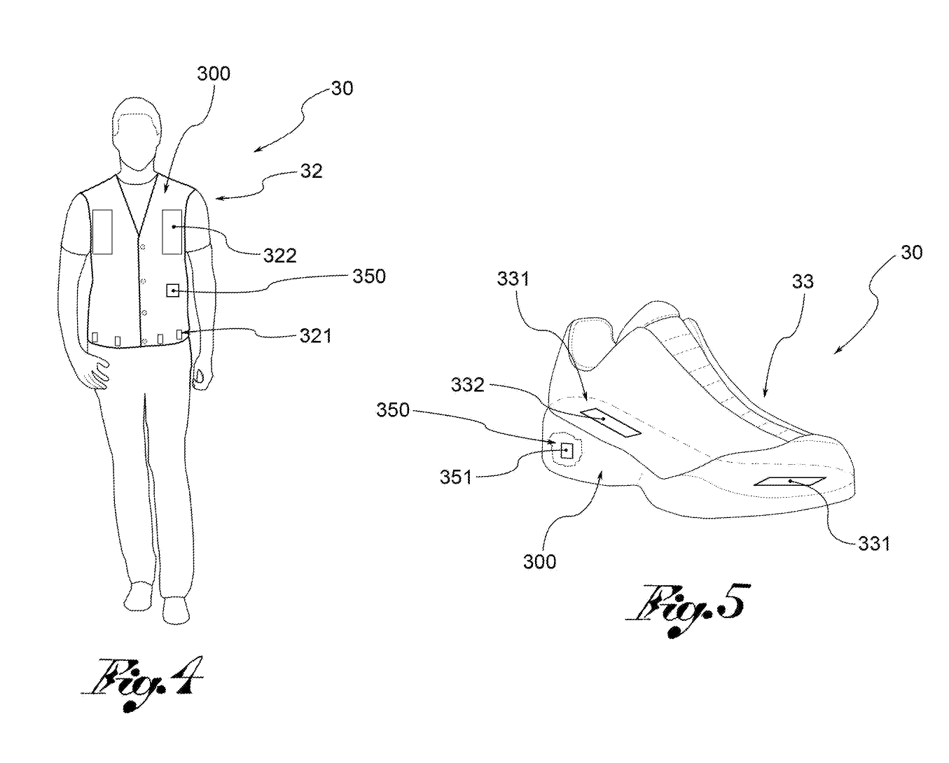

FIG. 5 shows a schematic diagram of an item of clothing, specifically a shoe, comprising a series of physical state and/or motor detection means, comprised in a preferred embodiment of the system which the present invention relates to;

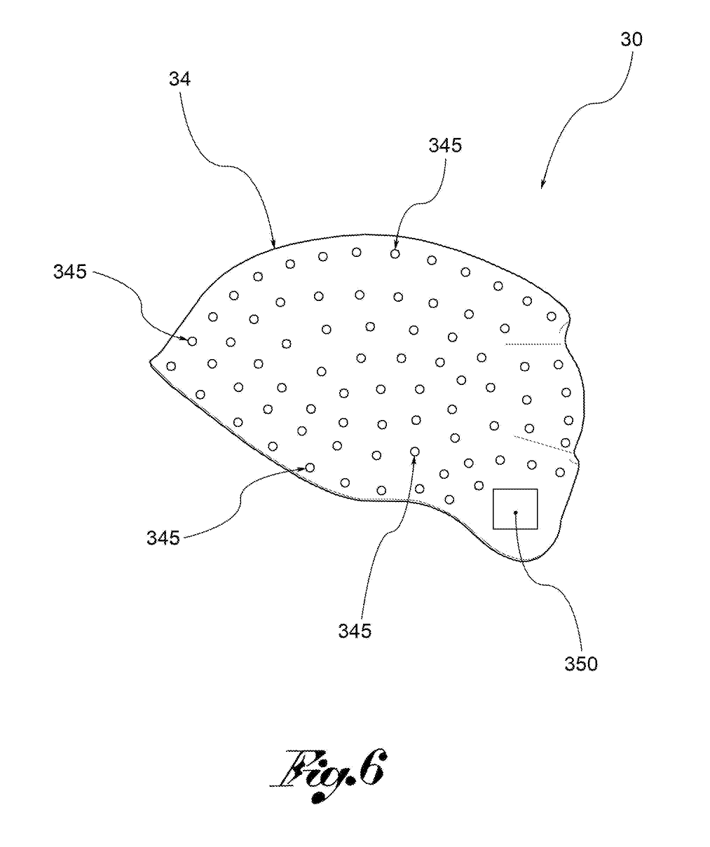

FIG. 6 shows a schematic diagram of an item of clothing, specifically, a helmet, comprising a series of physical state and/or motor detection means, comprised in a preferred embodiment of the system which the present invention relates to.

With reference to the appended drawings, reference numeral 1 globally denotes a detection system of the method of performing a shooting exercise with a weapon 10, according to the present invention.

In a preferred embodiment, the detection system 1 comprises a weapon 10 for performing the shooting exercise.

Preferably, said shooting exercise provides that the user hits a fixed or moving target. The system 1 which the present invention relates to is suitable for sports training or military training such as police training. In fact, the system 1 which the present invention relates to is not limited to a single type of shooting, but proves suitable for any type of Olympic sport or training activity which provides for a step of taking aim and a step of releasing the shot towards the target during the performance of the exercise. In addition, the system 1 is suitable for any type of shooting exercise both in which the user is in a fixed position and in the case in which the user is moving.

In a preferred embodiment, said weapon 10 is a firearm, such as a rifle or a gun, regardless of the type of propulsion used to expel the bullet, whether by means of gunpowder or compressed air; in further embodiment variants, the weapon 10 is a cold weapon such as a bow or a crossbow.

In one embodiment, the system 1 further comprises an environmental detection device 200 suitable for detecting the result of the shooting exercise.

In some embodiment variants, the environmental detection device 200 is suitable for detecting the result of the shooting exercise according to the type of weapon 10 used, for example by means of acoustic sensors able to hear the noise of the clay pigeon struck, or again visual type sensors suitable to detect the centre, and relative position of the shot fired, on the target. Furthermore, the environmental detection device 200 is also suitable for detecting the result of the shooting exercise; preferably, in fact, on the firing ranges and shooting areas, the error is signalled by an acoustic signal.

In addition, for example in the embodiment using firearms, the environmental detection device 200 is also suitable for detecting the number of shots fired during the shooting exercise, for example the number of shots needed to strike the clay pigeon. In other words, the environmental detection device 200 is also suitable for detecting the method of performance by which the result was achieved; seeing whether the clay pigeon was a first or second strike”, that is hit by the first shot fired or by the second one.

In a preferred embodiment, moreover, the environmental detection device 200 is also suitable for detecting the environmental characteristics of the place which the shooting exercise is performed in. Preferably, the environmental detection device 200 is also suitable for detecting weather conditions, for example, climatic conditions, temperature, humidity, wind intensity; or again to detect more specific characteristics of the place in which the exercise is performed, such as the noise or echo.

In other words, the environmental detection device 200 is in fact suitable, using dedicated means, to detect weather and or climatic data and information, relative to the place in which the shooting exercise is performed, or even characteristics of the place itself, for example the method of noise propagation, whether an indoor or outdoor firing range.

In a preferred embodiment, the system 1 further comprises at least one of the following:
weapon detection means 100; and
at least one item of clothing 30 comprising physical state and/or motor detection means 300.

Preferably, the weapon detection means 100 are positioned on board said weapon 10 and are suitable for detecting the position and spatial arrangement of the weapon 10 in the performance of the shooting exercise.

Preferably, the weapon detection means 100 comprise at least one accelerometer 101 suitable for detecting the position and spatial arrangement of the weapon 10 during the performance of the shooting exercise; this way the smoothness of the movement when performing the shooting exercise is assessable.

In a further embodiment, the weapon detection means 100 comprise at least one magnetometer 105 or electronic compass, by means of which the direction of the weapon is detectable and assessable.

This way the weapon detection means 100 are suitable to assess how the weapon 10 is positioned, held or griped by the user while performing the shooting exercise, either when taking aim or firing. In other words, the spatial position of the weapon 10 is detectable whether in static or dynamic conditions.

Preferably, the number and type of accelerometers 101 placed on the weapon 10 is variable so that a more accurate detection and simultaneous assessment of the position and of the spatial arrangement of the weapon 10, such as its inclination, proves possible.

Preferably, the accelerometers 101 are triaxial accelerometers.

In the embodiment of the system, in which the weapon 10 presents a support for the user, for example in the case in which the weapon is a rifle, the butt of said rifle, the weapon detection means 100 also comprise at least one pressure sensor 102. Preferably, the pressure sensor 102 is suitable for measuring the pressure intensity which the user applies to the weapon with cheekbone or cheek, or with the chest, or with the shoulder, depending on the type of shooting.

In a preferred embodiment, the firearm detection means 100 are provided in a kit for mounting on said weapon 10.

In a preferred embodiment, the system 1 comprises at least one item of clothing 30 worn by the user of the system 1 in such a way that his physical and/or motor activities are detected during the performance of the shooting exercise.

In particular, the item of clothing 30 comprises physical state and/or motor detection means 300 suitable for such purpose of detecting the physical and/or motor characteristics of the user wearing said garment 30.

In a preferred embodiment, the item of clothing 30 is a T-shirt or sweater 31, or in general an item of clothing suitable for adhering to the body, and in particular to the user's chest when worn.

Preferably, the T-shirt or similarly the sweater 31 is made of elasticised material so as to better adhere to the user's body.

In a preferred embodiment, the physical state and/or motor detection means 300 comprise a heart beat sensor 311 suitable for detecting the number and frequency of the user's heartbeats.

In a further embodiment, the physical state and/or motor detection means 300 comprise a breathing detector apparatus 312, suitable for detecting the user's breathing frequency and/or length of breath. Preferably, the breathing detector apparatus 312 comprises at least one strain gauge, suitable to detect the movement of the chest during the breathing movement. Preferably, the breathing detector apparatus 312 extends, in the form of a chest band, around the user's chest.

In a preferred embodiment, the physical state and/or motor detection means 300 comprise means of analysis of the position of the user's chest 313, whether static or dynamic, during performance of the shooting exercise, specifically suitable to detect the position and the movement of the user, for example by means of accelerometers.

In a preferred embodiment, the physical state and/or motor detection means 300 comprise one or more pressure sensors 314, suitable for detecting the eventual contact made by the user between the T-shirt or sweater 31 and the weapon 10; preferably, the pressure sensor 314 is located on the T-shirt or sweater 31 in such a way that it is positioned on the chest and/or on the shoulder when said T-shirt or sweater 31 is worn.

Preferably, the T-shirt or sweater 31 is suitable to comprise at least one of the aforementioned physical state and/or motor detection means 300.

In a preferred embodiment, the item of clothing 30 is a coat 32, such as a waistcoat or jacket; the coat 32 comprises physical state and/or motor detection means 300 which in turn comprise at least one of the means of analysis of the position of the user's chest 321 or pressure sensor 322.

Preferably, similarly to the embodiment variant with T-shirt or sweater 31, in the coat 32 too the means of analysis of the position of the user's chest 321 are suitable for detecting the position of the user, whether static or dynamic, during the performance of the shooting exercise, for example by means of accelerometers.

Again, similarly to the embodiment variant with T-shirt or sweater 31, in the coat 32 too one or more pressure sensors 322 are suitable for detecting the eventual contact made by the user between the coat 32 and weapon 10; preferably, the pressure sensor 322 is located on the coat 32 in such a way that it is positioned on the chest and/or on the shoulder when worn.

Preferably, the coat 32, is suitable to comprise at least one of the aforementioned physical state and/or motor detection means 300.

In yet a further preferred embodiment, the item of clothing 300 is a pair of shoes 33 and in this case the physical state and/or motor detection means comprise at least one specific posture sensor 331 suitable for detecting the position of the user's body in contact with the ground.

Preferably, the posture sensor 331 is thus suitable for detecting the position, and the posture of the user both in static and dynamic situations of the shooting exercise.

In particular in fact, the physical state and/or motor detection means 300 comprised in the pair of shoes 33 are suitable to detect and assess the position of the user's barycentre during the shooting exercise.

In a preferred embodiment, the posture sensor 331 comprises at least one heel element 332 placed in the insole of the shoe 33 in the support portion where the heel of the foot rests, and at least one tip element 333 placed in the insole of the shoe 33 in the support portion where the tip of the foot rests; both the heel element 332 and the tip element 333 are suitable for detecting the pressure caused by the user's weight and thus detecting the posture and the balance thereof.

In further embodiments, the posture sensor 331 is matrix of sensors, made on a sheet, having the shape of the insole of the shoe 33 so as to be suitable to assess the state of pressure all over the sole of the foot on the ground; in any case in said embodiment also, at least one tip element 333 and one heel element 332 may be identified on said matrix of sensors.

In yet a further preferred embodiment, the item of clothing 300 is a helmet 34 and in this case the physical state and/or motor detection means 300 comprise a plurality of electrodes 345 for detecting the electric activity of the user's brain during the performance of the shooting exercise.

In other words, the plurality of electrodes 345 is suitable for detecting the brain activity of the user during performance of the shooting exercise, substantially performing an electro-encephalogram during performance of the shooting exercise.

In a preferred embodiment, the system comprises a collect and memorisation unit 50 suitable to receive and store the information detected by the environmental device 20, by the weapon detection means 100 and/or by the physical state detection means 300.

Preferably, the collection and storage unit 50 is operatively connected to the weapon detection means 100 and/or to the physical state detection means 300 and/or to the environmental detection device 200 by means of wireless data transmission signals, for example in Wi-Fi, in Bluetooth, infrared or the like. In a further embodiment, the collection and storage unit 50 is operatively connected to the weapon detection means 100 and/or to the physical state detection means 300 and/or to the environmental detection device 200 by means of mobile phone transmission signals, via dedicated protocols such as GSM, GPRS, HDSPA, UMTS, LTE, EDGE or the like.

In a preferred embodiment, said collection and storage unit 50 is part of PC 51, tablet 52, or of a smartphone 53 or another device fitted with receiver means and memorisation means.

More generally, in some embodiments, the collection and storage unit 50 comprises a dedicated memory for the memorisation of the data and of the information, said memory and the information stored therein is then accessible for consultation via a PC 51, tablet 52, or a smartphone 53.

In some embodiment variants, the collection and storage unit 50 is physical support with a memory.

In other embodiment variants, the data collection and storage unit 50 is a virtual collection store of the information, for example a cloud 500, suitable to receive and store the information collected by the various means and devices described above.

In a preferred embodiment, the data collection and storage unit 50 comprises a reception device suitable to receive the data and to send it to said virtual store in a cloud 500; in a preferred embodiment the reception device is a mobile device, for example a tablet 52, or a smartphone 53. Preferably, in said embodiment, the weapon detection means 100, the physical state detection means 300 and the environmental detection device 200 are operatively connected to the data collection and storage unit 50 and, specifically, are operatively connected to the mobile device, such as the tablet 52 or the smartphone 53, which in turn are connected to the virtual or multimedia store. Preferably, the connection between the weapon detection means 100, the physical state detection means 300, environmental detection device 200 and mobile device is of the wireless type, for example via Bluetooth, Wi-Fi, infrared; the connection between the mobile device and multimedia store is via a mobile phone data network.

In other words, preferably, the weapon detection means 100, the physical state detection means 300 and the environmental detection device 200 in turn comprise data forwarding means 150, 250, 350 suitable for forwarding the data and information detected and for forwarding it to the data collection and storage unit 50 operatively connected. In a preferred embodiment, the data forwarding means 150, 250, 350 communicate with the storage unit 50 in wireless mode, for example via Bluetooth, Wi-Fi, infrared or data or mobile phone network.

In other embodiments, the data forwarding means 150, 250, 350 comprise a module for inserting special SIM cards for traffic on the mobile phone network.

In a preferred embodiment, for example wherein the item of clothing is a shoe 33, the data forwarding means 350 comprise a data transmission device 351 preferably positioned in the shoe 33 and in particular in the heel or sole thereof.

In a preferred embodiment, the system 1 comprise software suitable for organising and managing the data received and stored by the storage unit, preferably according to the result of the shooting exercise.

Said software is preferably suitable to show, catalogue and even divide the information stored in the collection and storage unit 50 according to the result of the shooting exercise detected by the environmental detection device 200.

In a preferred embodiment, said software can customised and operated by a user in such a way as to optimally manage the consultation of the results; for example, the software comprises a series of filters so as to simplify the management of the data collected.

Preferably, the software is suitable to store the data according to which device of the data detection system 1 provides it.

Preferably, the software comprises a series of machine languages specifically suited to detect and interpret the different data and different information sent to the collection and storage unit 50 by the various devices and various detection means described above.

In a preferred embodiment, said software is installable and runnable on said collection and storage unit 50; specifically, this is possible whatever the embodiment of the collection and storage unit 50 is. That is to say, said software is installable on the PC 51, tablet 52 or smartphone 53 forming part of said collection and storage unit 50 or suitable to access said collection and storage unit 50. Or again, said software is installable on the virtual store, for example in the cloud 500, and is manageable via the PC 51, tablet 52 or smartphone 53.

In some preferred embodiments, the software is manageable via the PC 51, tablet 52 or smartphone 53 by means of dedicated applications or “Apps” or programmes installed therein. Preferably, said applications or programmes may be developed by third parties. In further embodiments, said applications or said dedicated programmes is installable on the virtual store, or on the PC 51, or on the tablet 52 or on the smartphone.

Innovatively, the detection system which the present invention relates to enables the user to perform shooting training with feedback on the performance of the said shooting exercise, thereby overcoming the need, obligatory in the prior art, to train in the presence of a trainer.

Advantageously the system is suitable for detecting the result of the shooting exercise and if necessary further information relative to the physical, physiological and/or motor characteristics of the user during the performance of said shooting exercise.

In addition, advantageously, the system is suitable to detect environmental characteristics relative to the place where the shooting exercise is performed.

According to a further advantageous aspect, the system is suitable to detect the position, the spatial arrangement and the direction of the weapon during the shooting exercise and thus suitable to verify the correct gripping of the weapon, the relative movements of the user and the weapon, the smoothness of the movements, and the correct support of the weapon by the user's body, for example with the cheekbone, chest or shoulder. Advantageously this takes place by also assessing the variations of support between the weapon and the user's body, also evaluating the intensity thereof.

According to yet a further advantageous aspect, the system is suitable for detecting the physical state of the user during the shooting exercise, detecting his position, and/or correct posture and stance, his barycentre.

Advantageously the system is suitable for detecting the physical state of the user, detecting breathing frequency and/or heartbeat and/or brain activity. Advantageously the system proves even more useful than the presence of a trainer by picking up information on the user which cannot be perceived externally.

In addition, advantageously, the system is extremely accurate so as to permit an accurate assessment of the user's errors during performance of the shooting exercise, assessing for example if any errors in the shooting exercise are due to the positioning of the user's barycentre or his movement or physiological state.

According to a further advantageous aspect, the system is not limited to the type of weapon used nor to the type of activity performed; in addition the system proves extremely useful in both dynamic and static situations, that is to say whether the user is in a fixed position dung the exercise, or moving, for example from one shooting station to another.

Advantageously therefore the system is useful for both sports type training and military type training.

According to yet a further advantageous aspect, all the information is received and stored by means of the collection and storage unit.

Advantageously moreover, said collection and storage unit and/or the data memorised therein are consultable by means of a PC, a smartphone a tablet, in some embodiments, said storage unit is part of said PC, tablet or smartphone.

According to a further advantageous aspect, the collection and storage unit saves the data and information remotely for example in a virtual store, in a cloud.

Advantageously the consultation of the information collected and stored is simple to mange and use by the user by means of the software and/or applications and/or special programmes or “apps”.

According to a further advantageous aspect, the system is suitable to collect and store varied information according to the detection device or detection means providing it.

Advantageously the entire practice session can be reconstructed by means of the software, depending on the results and the data collected.

In addition, advantageously, the system is not limited to a certain number of items of clothing comprising physical state and/or motor detection means or weapon detection means but is functional and achieves its purpose using at least one of said means, improving its functioning as the number of said means used increases.

It is clear that a person skilled in the art may make modifications to the system described above so as to satisfy contingent requirements, for example by shifting some of the physical state and/or motor detection means provided for on one item of clothing to another, all contained within the scope of protection as defined by the following claims.

1. Detection system for performing a shooting exercise with a weapon, wherein the detection system comprises:
a weapon for performing the shooting exercise;
weapon detection means, positioned on board said weapon, suitable for detecting a position and spatial arrangement of the weapon in performance of the shooting exercise;
at least one item of clothing comprising physical state and/or motor detection means suitable for detecting physiological characteristics and/or motor characteristics of a user in the performance of the shooting exercise;
an environmental detection device suitable for detecting a result of the shooting exercise;
a collection and storage unit suitable for receiving and storing information detected by the weapon detection means and by the physical state and/or motor detection means and by the environmental device.

2. System according to claim 1, wherein the environmental detection device is suitable for detecting environmental characteristics, such as the weather and or climatic characteristics, relative to a place in which the shooting exercise is performed or the physical characteristics, such as noise propagation mode relative to the place in which the shooting exercise is performed.

3. System according to claim 1, wherein the weapon detection means comprise at least one accelerometer suitable for detecting position and spatial arrangement of the weapon during the performance of the shooting exercise.

4. System according to claim 1, wherein the weapon detection means comprise at least one magnetometer for detecting the direction of the weapon during the performance of the shooting exercise.

5. System according to claim 1, wherein the weapon for performing the shooting exercise is a rifle.

6. System according to claim 5, wherein the weapon detection means comprise at least one pressure sensor positioned on a butt of the rifle.

7. System according to claim 1, wherein the firearm for performing the shooting exercise is a gun.

8. System according to claim 1, wherein the item of clothing is a T-shirt or sweater, and the physical state and/or motor detection means comprise at least one of the following:
a) a heart beat sensor suitable for detecting number and frequency of the user's heartbeats;
b) a breathing detector apparatus suitable for detecting user's breathing frequency and/or length of breath;
c) means for analysing a position of the user's chest;
d) a pressure sensor suitable for detecting any weapon-T-shirt or sweater-contact.

9. System according to claim 1, wherein the item of clothing is a waistcoat or jacket, and the physical state and/or motor detection means comprise at least one of the following:
a) means of analysis of the position of the user's chest;
d) a pressure sensor suitable for detecting any weapon-T-shirt or sweater-contact.

10. System according to claim 1, wherein the item of clothing is a pair of shoes, and the physical state and/or motor detection means comprise at least one posture sensor suitable for detecting a position of the user's body in contact with the ground.

11. System according to claim 10, wherein the posture sensor comprises at least one heel element placed in an insole of the shoe in a support portion where the heel of the foot rests, at least one tip element placed in the insole of the shoe in the support portion where the tip of the foot rests, and a data transmission device, positioned in the heel, operatively connected to a collection and storage unit.

12. System according to claim 1, wherein the clothing element is a helmet and the physical state and/or motor detection means comprise a plurality of electrodes for recording electric activity of the user's brain during the performance of the shooting exercise.

13. System according to claim 1, wherein the collection and storage unit is operatively connected to the weapon detection means and/or to the physical state and/or motor detection means and/or to the environmental detection device by wireless data transmission signals.

14. System according to claim 1, comprising software suitable for organising all data received and stored by the collection unit according to the result of the shooting exercise.

15. System according to claim 1, wherein the collection and storage unit is a PC or a smartphone or a tablet.

16. System according to claim 1, wherein the collection and storage unit is operable by a PC, or a smartphone or a tablet.

17. System according to claim 1, wherein the collection and storage unit memorises data on a virtual store.
